(19) United States
(12) Patent Application Publication
(10) Pub. No.: US 2013/0171197 A1
(43) Pub. Date: Jul. 4, 2013

(54) HYALURONIC ACID HYDROGEL AND USE THEREOF

(71) Applicants: Mei-Ling Ho, Kaohsiung City (TW); Rajalakshmanan Eswaramoorthy, Kaohsiung City (TW); Shun-Cheng Wu, Kaohsiung City (TW); Gwo-Jaw Wang, Taipei City (TW); Je-Ken Chang, Kaohsiung City (TW); Yin-Chih Fu, Kaohsiung City (TW); Cherng-Chyi Tzeng, Kaohsiung City (TW); Chau-Zen Wang, Tainan City (TW)

(72) Inventors: Mei-Ling Ho, Kaohsiung City (TW); Rajalakshmanan Eswaramoorthy, Kaohsiung City (TW); Shun-Cheng Wu, Kaohsiung City (TW); Gwo-Jaw Wang, Taipei City (TW); Je-Ken Chang, Kaohsiung City (TW); Yin-Chih Fu, Kaohsiung City (TW); Cherng-Chyi Tzeng, Kaohsiung City (TW); Chau-Zen Wang, Tainan City (TW)

(73) Assignee: KAOHSIUNG MEDICAL UNIVERSITY, Kaohsiung City (TW)

(21) Appl. No.: 13/338,800

(22) Filed: Dec. 28, 2011

(51) Int. Cl. A61K 47/36; A61K 9/00

(52) U.S. Cl. 424/400; 514/772.1

(57) ABSTRACT

The present invention provides a hydrogel comprising a poly(N-isopropylacrylamide) cross-linked hyaluronic acid. The present invention also provides a method of synthesizing a hydrogel comprising poly(N-isopropylacrylamide) cross-linked hyaluronic acid, which comprises (a) synthesizing methacrylated hyaluronic acid; and (b) copolymerizing methacrylated hyaluronic acid with N-isopropylacrylamide.

### FIELD OF THE INVENTION

The present invention relates to a hydrogel comprising a poly(N-isopropylacrylamide) cross-linked hyaluronic acid, and a preparation method and use thereof.

### BACKGROUND OF THE INVENTION

Poly(N-isopropylacrylamide) (PNIPAM) is well known thermoresponsive polymer, exhibiting a lower critical solution temperature (LCST) around 32° C. in aqueous solution, in which they swell below the LCST and shrink above LCST in water. PNIPAM based thermoresponsive smart hydrogels are the current interest of research in orthopaedic tissue engineering. However, their poor biocompatibility is the major difficulty to attain the tissue engineering applications in clinical level.

In recent years, great attention has been paid especially for tissue engineering applications to the development of stimuli-responsive hydrogels with unique properties such as biocompatibility, biodegradability and biological functionality. They may be prepared by combining thermoresponsive polymers with natural based polymeric component, to form “smart hydrogels”. The approach of combining biopolymers with thermo-responsive material has received a particular importance in tissue engineering field since the resulted materials exhibit thermo-sensitive characters combined with other unique properties, such as good biocompatibility, mechanical strength, biodegradability, and/or differentiation induction of stem cells.

Among the extracellular matrix, hyaluronan (HA) is the main glycosaminoglycan in the mesenchyme during the early stage of chondrogenesis. Most importantly, HA is the major physiological component of the articular cartilage matrix, and is particularly abundant in synovial fluid.

Therefore, crosslinking of hyaluronic acid with PNIPAM through polymerization will enhance their efficiency in tissue engineering applications.

### BRIEF DESCRIPTION OF THE DRAWINGS

FIG. 1 shows photographic image of HA-PNIPAM hydrogel at different temperature (LCST analysis).

FIG. 2 shows SEM images of PNIPAM and HA-PNIPAM smart hydrogels.

FIG. 3 shows cell survival by Live and dead staining analysis on rADSCs encapsulated in PNIPAM or HA-PNIPAM at day 5. (Magnification: 200×)

FIG. 4 shows rADSCs encapsulated in PNIPAM or HA-PNIPAM. More cell aggregation shown in HA-PNIPAM group at day 5 and day 7. (Magnification: 200×)

FIG. 5A shows rADSCs encapsulated in PNIPAM or HA-PNIPAM higher GAG matrix formation shown in HA-PNIPAM group at 5 day and day 7 by Alcian blue staining (Magnification: 400×).

FIG. 5B shows the quantification result of cartilage nodules formed in PNIPAM or HA-PNIPAM group FIG. 5A.

FIG. 6 shows the cells and cellular matrix of the cartilaginous tissues from rADSCs encapsulated in PNIPAM or HA-PNIPAM in rabbit joint cavity after 3 weeks by H&E stain. (Magnification: 4×. 100×. 400×)

FIG. 7 shows the glycosaminoglycans (GAGs) formation of the cartilaginous tissues from rADSCs encapsulated in PNIPAM or HA-PNIPAM in rabbit joint cavity after 3 weeks by Safranin-O fast green stain. (Magnification: 4×. 100×. 400×)

FIG. 8 shows the type II collagen formation of the cartilaginous tissues from rADSCs encapsulated in PNIPAM or HA-PNIPAM in rabbit joint cavity after 3 weeks by immunohistochemical (IHC) stain. (Magnification: 4×. 100×. 400×)

### SUMMARY OF THE INVENTION

The present invention relates to a hydrogel comprising a poly(N-isopropylacrylamide) cross-linked hyaluronic acid(PNIPAM-HA), and a method of synthesizing a hydrogel comprising poly(N-isopropylacrylamide) cross-linked hyaluronic acid, which comprises (a) synthesizing methacrylated hyaluronic acid; and (b) copolymerizing methacrylated hyaluronic acid with N-isopropylacrylamide.

### DETAILED DESCRIPTION OF THE INVENTION

The present invention verifies that crosslinking of hyaluronic acid with PNIPAM through polymerization will enhance their efficiency in stem cell transplantation for cartilage tissue engineering. In the present invention, a novel fabrication method is invented to fabricate the thermoresponsive HA-PNIPAM smart hydrogel and their morphology, biocompatibility, cell transplantation/encapsulation efficiency, and their tissue engineering applications such as chondrogenic inducing potential on rabbit adipose derived stem cells (rADSCs) is studied.

The present invention emphasizes the application of HA-PNIPAM thermoresponsive hydrogel on cartilage tissue engineering. The present invention finds that the HA-PNIPAM hydrogel have honey comb like structure and HA copolymerization forms interpenetrating network (IPN) in PNIPAM. The LCST gelation test shows that copolymerization of HA with NIPAM does not have a substantial influence on the LCST of PNIPAM. The Live and dead cytotoxicity analysis image shows that the rADSCs encapsulated in HA-PNIPAM shows significantly higher cell survival and cell aggregation than those in PNIPAM. The Alcian blue staining analysis shows that the chondrogenic matrix formation is increased in HA-PNIPAM encapsulated with rADSCs. These results indicates that the copolymerization of HA on PNIPAM enhances their efficiency on cell survival and chondrogenic differentiation. The HA-PNIPAM hydrogel mimicking the HA-enriched extracellular matrices (ECM) provides a suitable microenvironment to enhance chondrogenesis in rADSCs. These results suggest that HA-PNIPAM thermoresponsive hydrogel may be the appropriate cell carrier to enhance chondrogenic differentiation in rADSCs for cartilage regeneration and stem cell based tissue engineering.

Therefore, the present invention provides a hydrogel comprising a poly(N-isopropylacrylamide) cross-linked hyaluronic acid. In an embodiment, The hydrogel is a honey comb like structure, which can be used as a cell carrier for enhancing chondrogenic differentiation in stem cells, preferably in rabbit adipose derived stem cells. In an embodiment, the hydrogel can be applied in cartilage regeneration. In an another embodiment, the hydrogel can be applied in stem cell based tissue engineering. In an embodiment, the hydrogel is a thermo-responsive hydrogel. Preferably, the gelation temperature of the hydrogel is at 34° C.

The present invention also provides a hydrogel comprising poly(N-isopropylacrylamide) cross-linked hyaluronic acid, which comprises (a) synthesizing methacrylated hyaluronic acid; and (b) copolymerizing methacrylated hyaluronic acid with N-isopropylacrylamide.

The present invention further provides a method of enhancing chondrogenic differentiation in stem cells, comprising culturing the stem cells with a hydrogel comprising poly(N-isopropylacrylamide) cross-linked hyaluronic acid. Preferably, the stem cells are rabbit adipose derived stem cells.

### EXAMPLES

The examples below are non-limiting and are merely representative of various aspects and features of the present invention.

### Example 1

Methods

Isolation and culture of rabbit adipose-derived stem cells (rADSCs): Subcutaneous adipose tissue was acquired from rabbits. The rADSCs were isolated from rabbit subcutaneous adipose tissue following the previously described method (Wu S C, Chang J K, Wang C K, Wang G J, Ho M L. Enhancement of chondrogenesis of human adipose derived stem cells in a hyaluronan-enriched microenvironment. Biomaterials 2010 February;31(4):631-640). The isolated rADSCs were cultured and expanded at 37° C. under 5% CO2 in K-NAC medium containing Keratinocyte-SFM (Gibco BRL, Rockville, Md.) supplemented with the EGF-BPE (Gibco BRL, Rockville, Md.), N-acetyl-L-cysteine, L-ascorbic acid 2-phosphate sequimagnesium salt (Sigma, St. Louis, Mo.) and 5% FBS (Wu S C, Chang J K, Wang C K, Wang G J, Ho M L. Enhancement of chondrogenesis of human adipose derived stem cells in a hyaluronan-enriched microenvironment. Biomaterials 2010 February;31(4): 631-640).

Synthesis of PNIPAM cross-linked Hyaluronic acid (HA-PNIPAM): The fabrication of HA-PNIPAM was the two stage process, first the synthesis of the methacrylated hyaluronic acid (HA-MA) by reported procedure and copolymerization of HA-MA with NIPAM. Briefly, 500 mg of NIPAM dissolved in 10 ml of distilled water and added with 1:5 ratio of HA-MA, followed by deareated with purging nitrogen for about 20 mins at room temperature. Then, the 100 uL of TEMED and 100 uL of Ammonium persulphate were added through syringe. The polymerizing mixture was kept under 0° C. for overnight and protected from light by wrapping silver foil. The formed polymer product was diluted by adding 10 mL of DDW, followed by vigorous dialysis for three days to remove the unreacted starting materials and lyophilized. The final HA-PNIPAM was stored at 4° C. till use.

The lower critical solution temperature (LCST) gel formation test: 5% w/v PBS solution of PNIPAM or HA-PNIPAM was used to test LCST gel formation under increasing temperature.

Scanning electron microscopy examination: The morphological characteristics of both PNIPAM and HA-PNIPAM hydrogels were observed by using scanning electron microscopy (SEM, JEOL, Tokyo, Japan). However, freeze dried samples were first coated with gold via a sputter-coater at ambient temperature. Micrographs of both scaffolds were taken 100×.

Cell encapsulation of rADSCs in PNIPAM or HA-PNIPAM hydrogel: 1×10⁶ cells/mL rADSCs suspension of 5% w/v PNIPAM or HA-PNIPAM in PBS were prepared. A 200 μL of cell-hydrogel suspension was added into each 24 well cell culture plate. The cultures were kept under 37° C. for 5 mins to form the gel. After gelation, 1 ml of pre-warmed fresh basal medium (basal medium: DMEM containing 10% FBS (Hyclone, Logan, Utah), 1% nonessential amino acids and 100 U/ml penicillin/streptomycin (Gibco-BRL, Grand Island, N.Y.) was added into each well. The medium was changed every 2 days. At every indicated time interval, cells/scaffold constructs were collected for further experimental analysis.

Cell survival in PNIPAM and HA-PNIPAM hydrogel carrier: The survivability of rADSCs in the PNIPAM and HA-PNIPAM hydrogel was assessed using a Live and dead cytotoxicity kit. Live/dead images of PNIPAM and HA-PNIPAM hydrogel constructs were taken 5 days after cells were encapsulated. The media was discarded and the constructs were washed twice with PBS. Cell survival was assessed based on the integrity of the cellular membrane using a Live and dead cytotoxicity Kit (Molecular Probes, Eugene, Oreg.), which contains calcein-AM (live dye, green) and ethidium homodimer-1 (dead dye, red). A dye solution was made with 0.5 μl of calcein-AM and 2 μl of ethidium homodimer-1 in 1 ml of the standard medium. A slice of the construct was incubated in 1 ml of the Live and dead dye solution in a 3.5 mm dish for 30 min. Fluorescence microscopy was performed using a fluorescein optical filter to detect calcein-AM and a rhodamine optical filter to detect ethidium homodimer-1.

Chondrogenic differentiation: The PNIPAM or HA-PNIPAM encapsulated with rADSCs were seeded in 24 well plates and kept in incubator at 37° C., 5%, CO2, for cultured for 5 and 7 days. The culture medium was changed every 2-3 days, After 5 and 7 days the cells were fixed by using 4% of the parafomardehyde and tested the chondrogenesis using Alcian blue staining.

Alcian blue stain: 0.5% Alcian blue at pH 1.0 was added to the each dish and stained over night. Wash twice with double distilled ultra-pure, chondroitin sulfate formed where dyed blue in color.

Dimethylmethylene blue (DMMB) assay: At every indicated time interval, cells/hydrogel constructs were collected and dissolved in 1 ml Triton (1 ml of Triton per sample). DNA content and sulfated glycosaminoglycan (sGAG) accumulation by cells was quantified spectrofluorometrically using 33258 Hoechst dye and dimethylmethylene blue (DMMB), respectively. Standard curve for DMMB assay was generated using aqueous chondroitin sulfate C (Sigma-Aldrich, St. Louis, Mo.) solution, with concentrations ranging from 0 to 25 μg/μl.

Histological analysis and immunostaining of cartilaginous tissue: The cartilaginous tissues from rADSCs cultured in PNIPAM or HA-PNIPAM in rabbit joint cavity were harvested after 3 weeks, and fixed with 10% neutral buffered formalin prior to histologic preparation. The samples were paraffin embedded, and 5-μm microsections were prepared. Histochemical and immunohistochemical analyses were concurrently employed to assess the microscopic changes in the cartilaginous tissues. Hematoxylin-eosin (H&E), Safranin-O fast green staining and Immunohistochemical (IHC) staining were brought up. The cells and cellular matrix of the cartilaginous tissues were observation with H&E. GAG was stained with Safranin-O fast green (1% Safranin O counterstained with 0.75% hematoxylin and then 1% fast green; Sigma). Localized type II collagen were immunostained. Sections were observed with a microscope at 4×, 100× and 400×. At 400× magnification, the central and the edge of area was compared with that of the control group. IHC staining for type II collagen was performed as follows. The optimal condition for enzyme digestion for type II collagen immunostaining was a mixture of 2.5% hyaluronidase and 1 mg/ml of Pronase in PBS (pH 7.4; Sigma) at 37° C. for 1 hour. Sections were then blocked with FBS for 1 hour and incubated with primary antibodies to type II collagen (mouse monoclonal antibody; Chemicon, Temecula, Calif.) at 37° C. for 4 hours. The secondary antibodies were incubated for 30 minutes using biotin-labeled goat anti-mouse immunoglobulin for type II collagen (Dako, Carpinteria, Calif.) and horseradish peroxidase-conjugated streptavidin (Biocare Medical). Staining with a 3,3′-diaminobenzidine solution containing 0.01% hydrogen peroxide resulted in a brown color. Finally, sections were counterstained with hematoxylin and observed with a microscope.

Statistical analysis: Three independent cultures for biochemical analysis were tested. Each experiment was repeated at least three times, and data (expressed as mean±SEM) from a representative experiment were shown. Statistical significance was evaluated by one-way analysis of variance (ANOVA), and multiple comparisons were performed by Scheffe's method. A p<0.05 was considered significant.

### Results

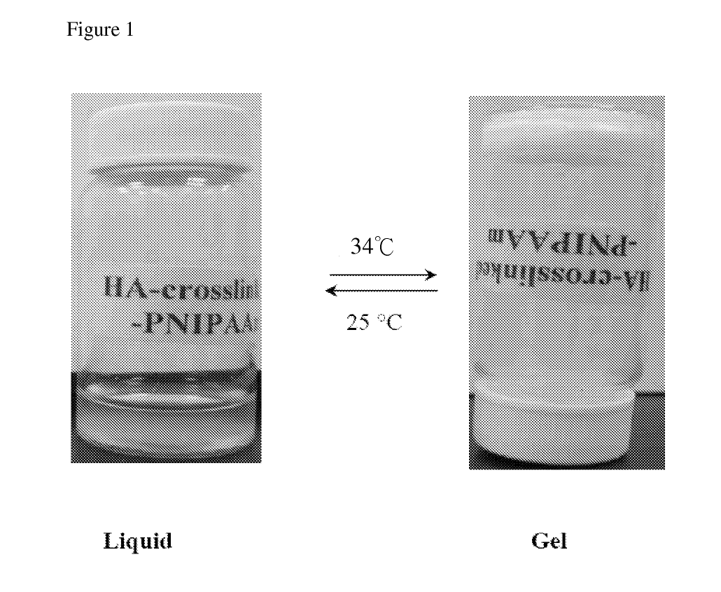

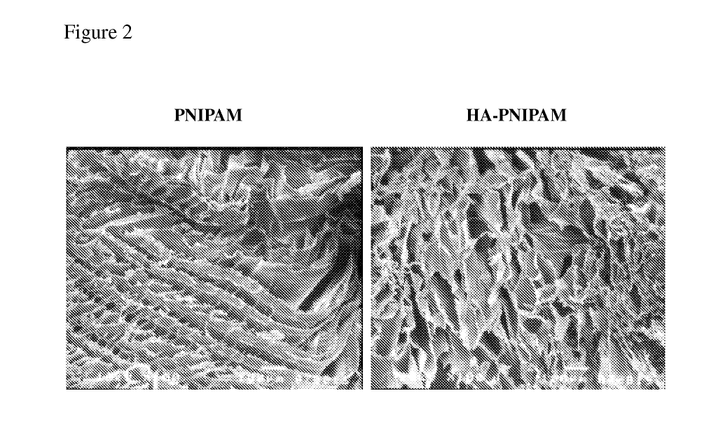

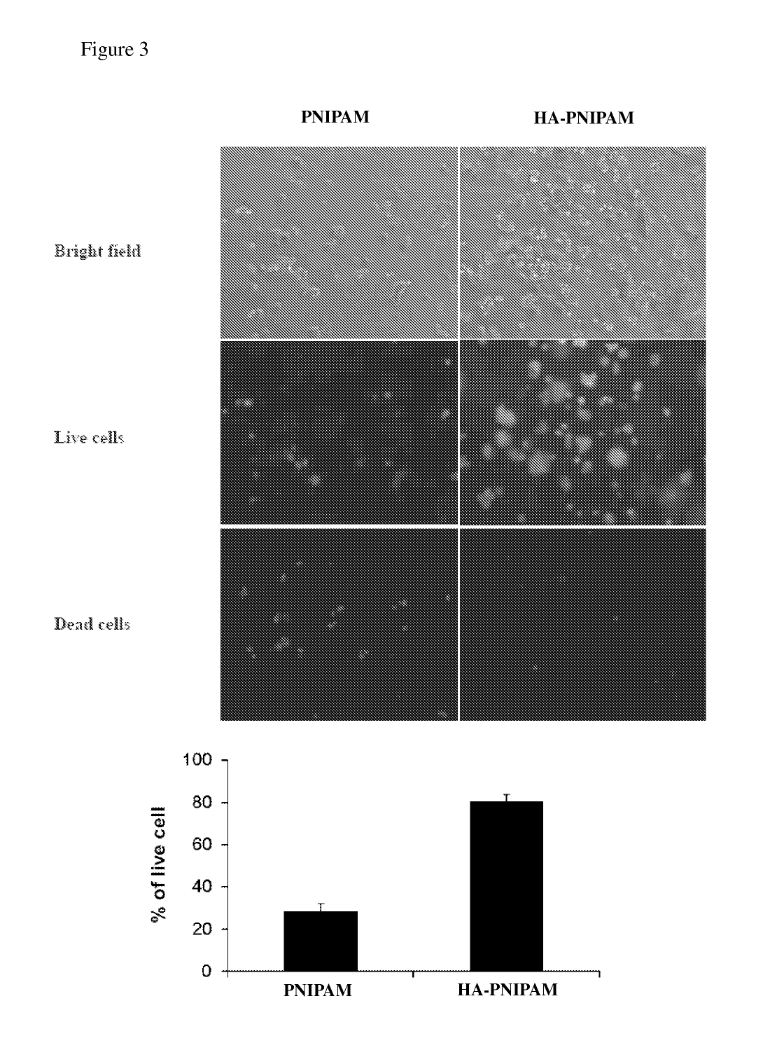

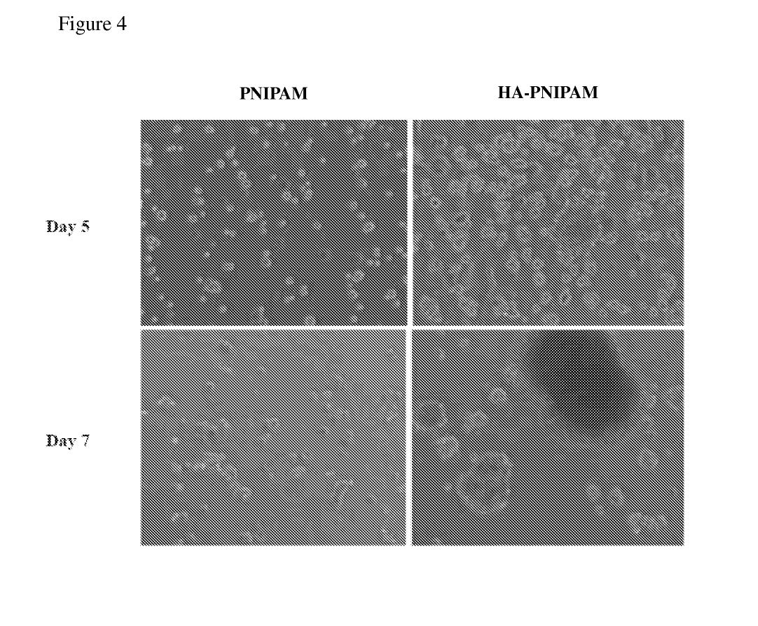

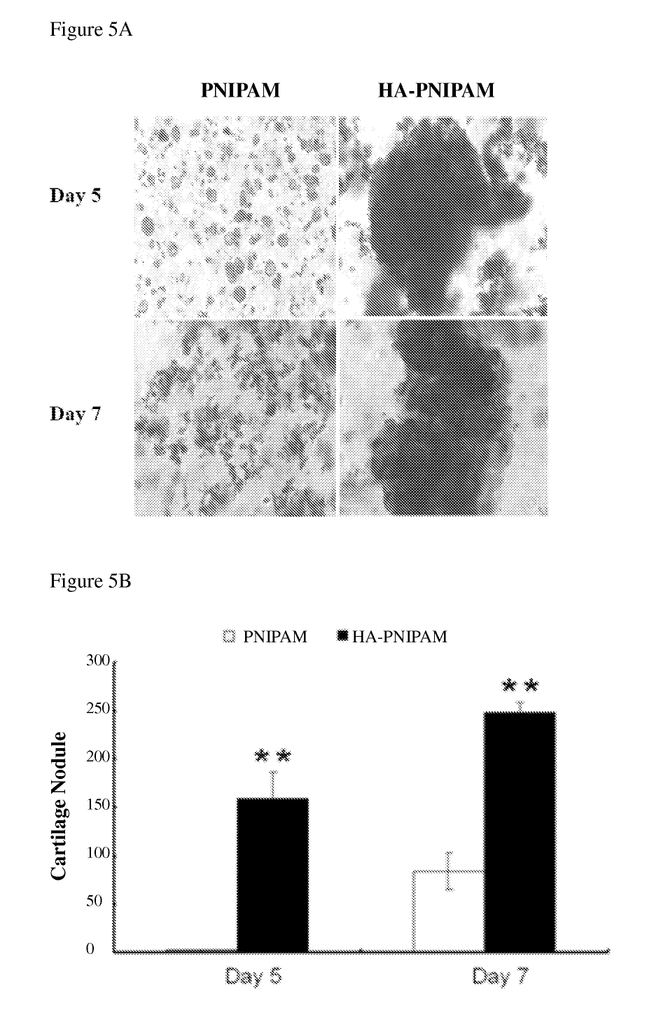

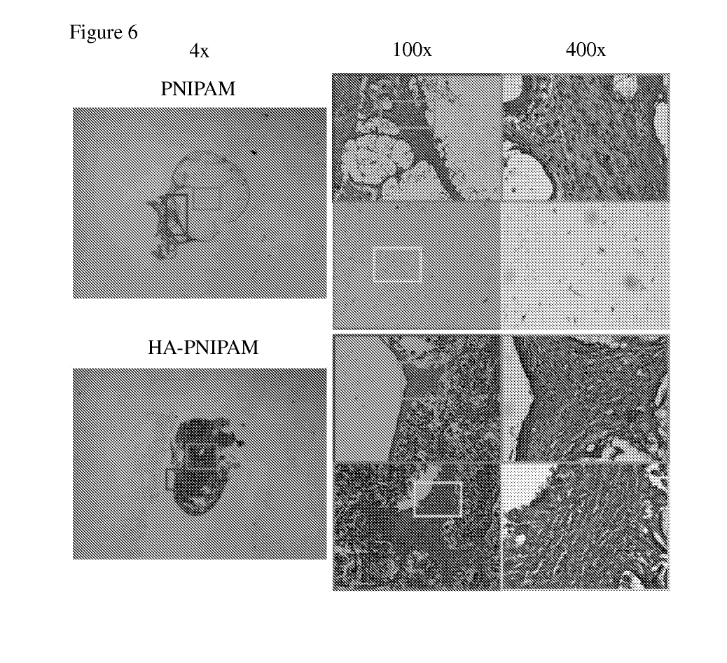

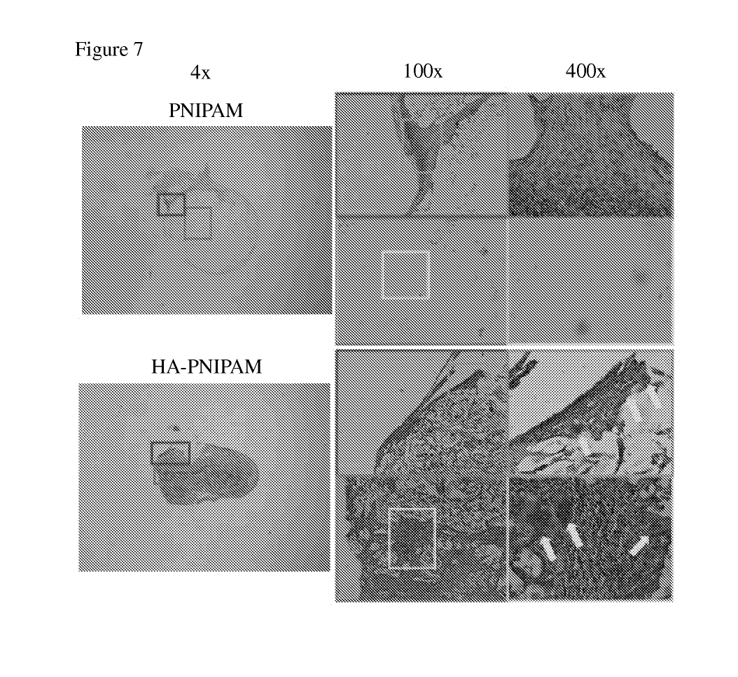

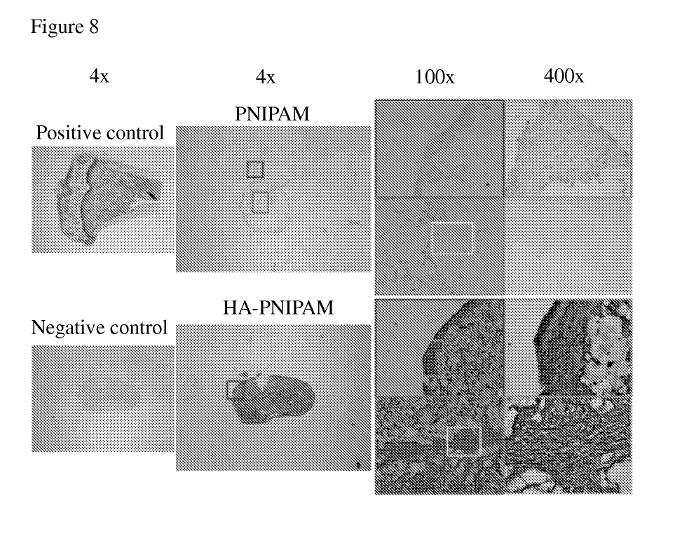

The LCST analysis showed that the gelation temperature for HA-PNIPAM hydrogel was 34° C. (FIG. 1). The SEM observation on HA-PNIPAM showed that the HA forms the interpenetrating network into PNIPAM with honey comb like structure (FIG. 2). The HA-PNIPAM hydrogel had 97±2% cell encapsulation efficiency. The survival rate test, we employed rADSCs seeded in PNIPAM and HA-PNIPAM for 5 days. We found that HA-PNIPAM exhibited a higher survival rate (over 80%) than PNIPAM (<40%) (FIG. 3). The HA-PNIPAM hydrogel encapsulated rADSCs showed more cell aggregation ability at day 5 and 7 (FIG. 4). The Alcian blue staining showed that HA-PNIPAM hydrogel cultured rADSCs formed more cartilaginous matrix compared to PNIPAM at day 5 and 7 (FIG. 5). In vivo study, the rADSCs were cultured in PNIPAM and HA-PNIPAM hydrogel and injected into joint cavity, we found that more matrix were formed in HA-PNIPAM group by H&E staining (FIG. 6), and higher expression level of GAG and type II collagen detected by Safranin-O fast green staining (FIG. 7) and Immunohistochemical (IHC) staining (FIG. 8), respectively.

One skilled in the art readily appreciates that the present invention is well adapted to carry out the objects and obtain the ends and advantages mentioned, as well as those inherent therein. The hydrogels, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A hydrogel comprising a poly(N-isopropylacrylamide) cross-linked hyaluronic acid.

2. The hydrogel of claim 1, which is a honey comb like structure.

3. The hydrogel of claim 1, which is a cell carrier for enhancing chondrogenic differentiation in stem cells.

4. The hydrogel of claim 3, wherein the stem cells are mammalian adipose derived stem cells.

5. The hydrogel of claim 1, which is applied in cartilage regeneration.

6. The hydrogel of claim 1, which is applied in stem cell based tissue engineering.

7. The hydrogel of 1, which is a thermo-responsive hydrogel.

8. The hydrogel of 1, wherein the gelation temperature of the hydrogel is at 34° C.

9. A method of synthesizing a hydrogel comprising poly(N-isopropylacrylamide) cross-linked hyaluronic acid, which comprises (a) synthesizing methacrylated hyaluronic acid; and (b) copolymerizing methacrylated hyaluronic acid with N-isopropylacrylamide.
